(19) United States
(12) Patent Application Publication
(10) Pub. No.: US 2025/0049874 A1
(43) Pub. Date: Feb. 13, 2025

(54) RHODIOLA ROSEA NANOEMULSION AND PREPARATION METHOD THEREOF

(71) Applicant: MD RESEARCH LTD, Denver, CO (US)

(72) Inventors: Benni Iskandar, Pekanbaru City (ID); Ching-Peng Wei, Taipei City (TW); Ching Kuo Lee, New Taipei City (TW)

(73) Assignee: MD RESEARCH LTD, Denver, CO (US)

(21) Appl. No.: 18/231,335

(22) Filed: Aug. 8, 2023

(51) Int. Cl. A61K 36/41; A61K 9/107; A61K 31/7034; A61K 47/10; A61K 47/26; A61K 47/44

(52) U.S. Cl. CPC A61K 36/41; A61K 9/1075; A61K 31/7034; A61K 47/10; A61K 47/26; A61K 47/44; A61K 2236/333

(57) ABSTRACT

Provided is a Rhodiola rosea nanoemulsion, comprising Rhodiola rosea extract, surfactant, cosurfactant and oil. The surfactant includes diethylene glycol monoethyl ester or polysorbate 80, the cosurfactant includes polysorbate 80, sorbitan oleate 80, or caprylocaproyl polyoxyl-8 glycerides (Labrasol®), and the oil includes Labrafac™ oil or soybean oil, and Rhodiola rosea extract coated in oil-in-water form. Also provided is a method for preparing the Rhodiola rosea nanoemulsion, so as to provide the Rhodiola rosea nanoemulsion suitable for drug delivery.

### BACKGROUND

### Field of Invention

The present invention relates to nanoemulsion and preparation method thereof. More particularly, the present invention relates to Rhodiola rosea nanoemulsion and preparation method thereof.

### Description of Related Art

Nanoemulsions are one of the drug delivery system in dosage forms used to deliver the active ingredient to the target site in our body. Nanoemulsion have consists of oil phase, water and surfactant (cosurfactant). The droplet of particle size of nanoemulsions is in nano range varying, start from 10 to 1000 nm. Nanoemulsion with the particle size in below 500 nm will have clear emulsion and good stability. The Brownian motion style of small droplet size is therefore enough to overcome their low gravitational properties resulting with good physical stability, easy to penetrate and have characteristic like a clear solutions. Nanoemulsions can be generally classified into water-in-oil (W/O) and oil-in-water (O/W) types.

Rhodiola rosea is also known as “golden root”. Traditionally known as a plant that is powerful enough to fight viral infections, relieve depression, lose weight, increase energy, and even improve sexual function as well. Rhodiola rosea root has a wide range of pharmacological activities, including neuroprotection, anti-inflammation, anti-aging, cardioprotection, anti-cancer and antioxidation. Rhodiola rosea have any secondary metabolites including flavonoids, phenylethanoids, phenylpropanoids, flavolignans, phenolic acids, essential oil, plysaccaharides and also have cyanogenic glucosides.

However, Rhodiola rosea have great potential in cosmetic and pharmaceutical industries due to the contains a number of pharmacological characteristics, including antioxidant activity.

### SUMMARY

The present disclosure provides a Rhodiola rosea nanoemulsion, comprising Rhodiola rosea extract; a surfactant comprising diethylene glycol monoethyl ester or polysorbate 80; a cosurfactant comprising polysorbate 80, sorbitan oleate 80, or caprylocaproyl polyoxyl-8 glycerides; and an oil comprising medium-chain triglycerides (propylene glycol dicaprylocaprate, also called Labrafac™ oil) or soybean oil, and the Rhodiola rosea extract coated in oil-in-water nanoemulsion form.

In some embodiments, the Rhodiola rosea extract comprises Rhodiola rosea root ethanol extract.

In some embodiments, the Rhodiola rosea extract comprises 1,2,3,4,6-penta-O-galloyl-β-D-glucopyranoside and 1,2,3,6-tetra-O-galloyl-4-O-p-hydroxybenzoyl-β-D-glucopyranoside.

In some embodiments, the surfactant has a weight percentage from 10% to 30%; and the cosurfactant has a weight percentage from 10% to 30%, based on a total weight (100% by weight) of the Rhodiola rosea nanoemulsion.

In some embodiments, the surfactant being diethylene glycol monoethyl ester has a weight percentage from 10% to 30%; the cosurfactant being polysorbate 80 has a weight percentage from 10% to 30%, and the oil being propylene glycol dicaprylocaprate has a weight percentage from 1% to 5%, based on a total weight of the Rhodiola rosea nanoemulsion.

In some embodiments, the weight percentage of the surfactant is 10%, and the weight percentage of the cosurfactant is 16.63%.

In some embodiments, the surfactant being polysorbate 80 has a weight percentage from 10% to 30%; the cosurfactant being sorbitan oleate 80 has a weight percentage from 10% to 30%, and the oil being soybean oil has a weight percentage from 1% to 5%, based on a total weight of the Rhodiola rosea nanoemulsion.

In some embodiments, the weight percentage of the surfactant is 10%, and the weight percentage of the cosurfactant is 29.87%.

In some embodiments, the surfactant being diethylene glycol monoethyl ester has a weight percentage from 10% to 30%; the cosurfactant being caprylocaproyl polyoxyl-8 glycerides (Labrasol®) has a weight percentage from 10% to 30%, and the oil being propylene glycol dicaprylocaprate has a weight percentage from 1% to 5%, based on a total weight of the Rhodiola rosea nanoemulsion.

In some embodiments, the weight percentage of the surfactant is 28.41%, and the weight percentage of the cosurfactant is 16.63%.

In some embodiments, the surfactant being diethylene glycol monoethyl ester has a weight percentage from 10% to 30%; the cosurfactant being polysorbate 80 has a weight percentage from 10% to 30%, and the oil being soybean oil has a weight percentage from 1% to 5%, based on a total weight of the Rhodiola rosea nanoemulsion.

In some embodiments, the weight percentage of the surfactant is 29.61%, and the weight percentage of the cosurfactant is 30%.

In some embodiments, the Rhodiola rosea nanoemulsion has a size from 10 nm to 500 nm, a polydispersity index (PDI) from 0.173 to 0.667, and a zeta potential from −96.7 mV to −7.39 mV.

The present disclosure also provides a method for preparing Rhodiola rosea nanoemulsion, comprising: providing a Rhodiola rosea extract; mixing the Rhodiola rosea extract and an oil to obtain an oil phase component; mixing a surfactant and a cosurfactant to obtain a water phase component; adding the oil phase component into the water phase component to form oil-in-water until emulsified to obtain the Rhodiola rosea nanoemulsion, wherein the surfactant comprises diethylene glycol monoethyl ester or polysorbate 80, wherein the cosurfactant comprises polysorbate 80, sorbitan oleate 80, or caprylocaproyl polyoxyl-8 glycerides; wherein the oil comprises propylene glycol dicaprylocaprate or soybean oil, wherein a combination that when the surfactant is the diethylene glycol monoethyl ester, the cosurfactant is the sorbitan oleate 80, and the oil is soybean oil is excluded.

In some embodiments, the step of adding the oil phase component into the water phase component comprises dropping the oil phase component into the water phase component.

In some embodiments, in the step of adding the oil phase component into the water phase component, forming oil-in-water nanoemulsion until emulsified is by spontaneous emulsification method (ultrasonic vibration).

In some embodiments, the surfactant has a weight percentage from 10% to 30%; and the cosurfactant has a weight percentage from 10% to 30%, based on a total weight of the Rhodiola rosea nanoemulsion.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows:

FIGS. 1A to 1G are effect of contour plots and three-dimensional graphs according to some embodiments of the present disclosure, showing effect of independent variables on globule size (Y1), PDI (Y2) and zeta potential (Y3) for Group F. FIGS. 1A, 1B and 1C are effect of surfactant and cosurfactant concentration on globule size, PDI and zeta potential in contour plots. FIG. 1D is desirability. FIG. 1E, 1F and 1G are effect of surfactant and cosurfactant concentration on globule size, PDI and zeta potential in 3D graphs of Group F.

FIGS. 2A to 2G are effect of contour plots and three-dimensional graphs according to some embodiments of the present disclosure, showing effect of independent variables on globule size (Y1), PDI (Y2) and zeta potential (Y3) for Group G. FIGS. 2A, 2B and 2C are effect of surfactant and cosurfactant concentration on globule size, PDI and zeta potential in contour plots. FIG. 2D is desirability. FIGS. 2E, 2F and 2G are effect of surfactant and cosurfactant concentration on globule size, PDI and zeta potential in 3D graphs of Group G.

FIGS. 3A to 3G are effect of contour plots and three-dimensional graphs according to some embodiments of the present disclosure, showing effect of independent variables on globule size (Y1), PDI (Y2) and zeta potential (Y3) for Group H. FIGS. 3A, 3B and 3C are effect of surfactant and cosurfactant concentration on globule size, PDI and zeta potential in contour plots. FIG. 3D is desirability. FIGS. 3E, 3F and 3G are effect of surfactant and cosurfactant concentration on globule size, PDI and zeta potential in 3D graphs of Group H.

FIGS. 4A to 4G are effect of contour plots and three-dimensional graphs according to some embodiments of the present disclosure, showing effect of independent variables on globule size (Y1), PDI (Y2) and zeta potential (Y3) for Group J. FIGS. 4A, 4B and 4C are effect of surfactant and cosurfactant concentration on globule size, PDI and zeta potential in contour plots. FIG. 4D is desirability. FIGS. 4E, 4F and 4G are effect of surfactant and cosurfactant concentration on globule size, PDI and zeta potential in 3D graphs of Group J.

FIGS. 5A to 5D are line charts of pH value for Group F (FIG. 5A), G (FIG. 5B), H (FIG. 5C) and J (FIG. 5D) in day 1, 30, 60 and 90 under 25° C., 40° C. and 4° C. according to some embodiments of the present disclosure.

FIGS. 6A to 6D are TEM photomicrographs with scale 500 nm for optimization formula. FIG. 6A: Optimization Formula F (Opt F), FIG. 6B: Optimization Formula G (Opt G), FIG. 6C: Optimization Formula H (Opt H), and FIG. 6D: Optimization Formula J (Opt J).

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

As used herein, “cosurfactant” is a chemical moiety that further reduces the surface tension of a liquid when used in combination with a surfactant.

As used herein, “spontaneous method” refers to that in spontaneous emulsification, the organic phase that is constituted by oil, surfactant, and cosurfactant is simply added to the aqueous phase with mild agitation. The surfactant present in the organic phase has a high affinity for the continuous phase. Therefore, upon mixing of two phases (organic or dispersed phase and aqueous or continuous phase), turbulence is produced and surfactant diffuses swiftly towards the aqueous phase and forms covering/film around the dispersed oil droplet by lowering the interfacial tension resulting in the spontaneous formation of nanoemulsion system. Cosurfactant further aids in causing the turbulence, lowering the interfacial tension between the two immiscible phases and easing the formation of dispersion by lodging in the unoccupied places around dispersed oil droplets that are left unguarded by surfactant molecules. The screening of surfactants for the spontaneous emulsification is based on the hydrophilic-lipophilic balance (HLB) which is the strength and the size of the hydrophilic and lipophilic moieties of the surfactant molecule.

As used herein, “Rhodiola rosea (RR)” contains about 140 chemical compounds in the subterranean portions. RR roots contain phenols, rosavin, rosin, rosarin, organic acids, terpenoids, phenolic acids and their derivatives, flavonoids, anthraquinones, alkaloids, tyrosol, and salidroside.

In some embodiments, the Rhodiola rosea nanoemulsion includes Rhodiola rosea extract, surfactant, cosurfactant, and oil.

In some embodiments, based on a total weight (100% by weight) of the Rhodiola rosea nanoemulsion, surfactant which is diethylene glycol monoethyl ester has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; cosurfactant which is polysorbate 80 has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; oil which is propylene glycol dicaprylocaprate has a weight percentage from 1% to 5%, for example, 2%, 3%, 4%, or any value between any two of these values.

In some embodiments, based on a total weight (100% by weight) of the Rhodiola rosea nanoemulsion, surfactant which is polysorbate 80 has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; cosurfactant which is sorbitan oleate 80 has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; oil which is soybean oil has a weight percentage from 1% to 5%, for example, 2%, 3%, 4%, or any value between any two of these values.

In some embodiments, based on a total weight (100% by weight) of the Rhodiola rosea nanoemulsion, surfactant which is diethylene glycol monoethyl ester has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; cosurfactant which is caprylocaproyl polyoxyl-8 glycerides (Labrasol®) has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; oil which is propylene glycol dicaprylocaprate has a weight percentage from 1% to 5%, for example, 2%, 3%, 4%, or any value between any two of these values.

In some embodiments, based on a total weight (100% by weight) of the Rhodiola rosea nanoemulsion, surfactant which is diethylene glycol monoethyl ester has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; cosurfactant which is polysorbate 80 has a weight percentage from 10% to 30%, for example, 11%, 12%, 13%, 14%, 15%, 16%, 17%, 18%, 19%, 20%, 21%, 22%, 23%, 24%, 25%, 26%, 27%, 28%, 29%, 30%, or any value between any two of these values; oil which is soybean oil has a weight percentage from 1% to 5%, for example, 2%, 3%, 4%, or any value between any two of these values.

A number of examples are provided herein to elaborate the RR nanoemulsion of the instant disclosure. However, the examples are for demonstration purpose alone, and the instant disclosure is not limited thereto.

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

For clarity, features and elements that are known in the art and are not essential to an understanding of the principles described will be omitted.

### Preparation Example

Surfactant, Cosurfactant and Oil phase is a crucial part of the preparation of Nanoemulsion formulation. The surfactant and cosurfactant were selected based on the emulsification abilities in nanoemulsion formation. The preparation of emulsification ability was used spontaneous method. The surfactant and cosurfactant was selected with differences of hydrophylic-lipophylic balance (HLB) combination. Transcutol® HP (diethylene glycol monoethyl ether), tween 80, span 80 and Labrasol® were used as a surfactant and cosurfactant. HLB values from (3-6) are more likely to produce water in oil (w/o) nanoemulsion, and oil in water (o/w) type is more likely to be formed in the range of (8-18). For the oil phase used different of carbon chain length (C8-C18) were utilized for the preparation of nanoemulsion through the spontaneous emulsification method. The selected oils are Labrafac™ and soybean oil as an oil phase.

After roots of Rhodiola rosea were dried, crush them with a grinder. The crushed roots of Rhodiola rosea were soaked and extracted with 95% ethanol at a weight-to-volume ratio of 1:20 (w/v) for 1 week to obtain an extract. The extract is concentrated by a vacuum concentrator to obtain dry Rhodiola rosea crude extracts. The above ethanol extraction step was repeated three times, and the three times' concentrated extracts were combined to obtain a Rhodiola rosea root ethanol extract.

After roots of Rhodiola rosea were extracted by ethanol, a voucher specimen (No. TMU060615) was identified and was deposited at the College of Pharmacy, Taipei Medical University, Taipei, Taiwan. The Rhodiola rosea root ethanol extract at least includes 1,2,3,4,6-penta-O-galloyl-β-D-glucopyranoside and 1,2,3,6-tetra-O-galloyl-4-O-p-hydroxybenzoyl-β-D-glucopyranoside. In this preparation for nanoemulsion formulation were prepare using low energy nanoemulsion preparation method. The nanoemulsions were prepared in the following steps: (1) Rhodiola rosea extraction and oil (also called essential oil) were mixed to obtain an oil phase component. (2) A surfactant and a cosurfactant were mixed for 15 minutes in 500 rpm, to obtain a water phase component. (3) The oil phase component was dropped into the water phase component and stirred for 60 minutes in 1000 rpm at 25° C., then mixed using sonicator for 15 minutes at 25° C. to form oil-in-water, and then let stand for 15 minutes until emulsified (spontaneous emulsification method) to obtain the Rhodiola rosea nanoemulsion. The Rhodiola rosea nanoemulsion was stored in not higher than 25° C.

### Example 1 Oil Phase Selection Test of Rhodiola Rosea Nanoemulsion

Please refer to table 1 below, test formulas are divided into group F, group Fx and group Fy. The difference of each group of the test formulas lies in the choice of oil phase. The group F is Labrafac®, the group Fx is corn oil, and the group Fy is sunflower oil. After the preparation was completed through the preparation example, the results showed that the test formula group F was clear and the particle size was 19.13 nm. As for the group Fx and group Fy, both were turbid and the particle size was greater than 1000 nm. In other words, when sunflower oil or corn oil is used as the oil phase formula of Rhodiola rosea nanoemulsion, the effects of clarification and nanoparticles will not be achieved.

TABLE 1

| Function | Group F | Group Fx | Group Fy | ratio |
|---|---|---|---|---|
| Active | Rhodiola rosea | Rhodiola rosea | Rhodiola rosea | 1% |
| ingredient | root ethanol | root ethanol | root ethanol | |
| | extract | extract | extract | |
| Surfactant | Transcutol ® | Transcutol ® | Transcutol ® | 30% |
| Cosurfactant | Tween 80 | Tween 80 | Tween 80 | 10% |
| Oil phase | Labrafac ® | Corn oil | Sunflower oil | 3% |
| Emulsifier | Ethanol | Ethanol | Ethanol | 10% |
| Buffer | Na. EDTA | Na. EDTA | Na. EDTA | 0.20% |
| Preservative | Na. | Na. | Na. | 0.30% |
| | Metabisulfit | Metabisulfit | Metabisulfit | |
| Water phase | Distilled water | Distilled water | Distilled water | Replenish |
| | | | | to |
| | | | | 100% |

### Example 2 Design for RR Nanoemulsions

Experimental design for formulation using response surface methodology (RSM) to test the effects of the percentage of surfactant and cosurfactant and two oils with same percentage. The combination as an independent variables surfactant (10-30%) cosurfactant (10-30%) separate into 4 groups, Group F (Transcutol®, Tween 80, Labrafac™). Group G (Tween 80, Span 80, soybean oil), Group H (Transcutol®, Labrasol®, Labrafac™) and Group J (Transcutol®, Tween 80, soybean oil). Three factor central composite design (CCD) was employed to evaluate the effects of the surfactant cosurfactant combinations on three dependent variables (responses variables), such as particle size, polydispersity index (PDI) and zeta potential. The particle size and polydispersity index were measured by dynamic light scattering (DLS), and the zeta potential was measured by Zetasizer. The Design Expert software® (Stat-Ease, Inc., Minneapolis, USA) was used for the experimental design and data analysis. Total of 13 runs experiments of formulation each group were generated (as show in Tables 2, 3, 4, and 5). The results were analyzed statistically and used for calculation for nanoemulsion with the minimum particle size, lowest PDI and optimum zeta potential for a check the characterization of nanoemulsion. The analysis was conducted at 25° C. with backscattering angle of 173°.

TABLE 2
Independent factors, dependent factors (responses) and parameters.

| Serial number | |
|---|---|
| | Independent variables factors |
| X1 | Surfactant 10-30% (% w/w) |
| X2 | Cosurfactant 10-30% (% w/w) |
| | Dependent variables (responses) |
| Y1 | Globule Size |
| Y2 | Polydispersity index |
| Y3 | Zeta potential |
| | Formulation parameters which were keep constant |
| Z1 | Stirring time 60 minutes |
| Z2 | Essential oil concentration 3% (% w/w) |
| Z3 | RR EtOH extract and Other ingredients |
| | concentration (% w/w) |

*1 w/w % Rhodiola rosea root ethanol extract, 10 w/w % ethanol (as an emulsifier), 0.2 w/w % Na. EDTA (as a buffer), 0.3 w/w % Na. Metabisulfit (as a preservative), and water replenish to 100% w/w % (based on total weight of the Rhodiola rosea nanoemulsion).

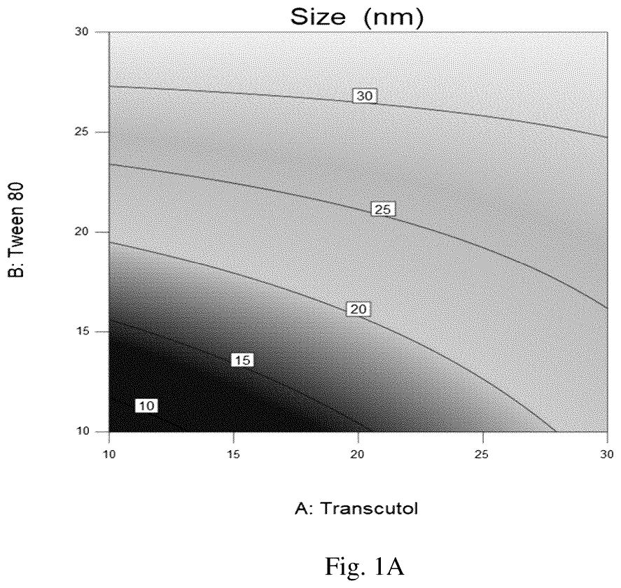

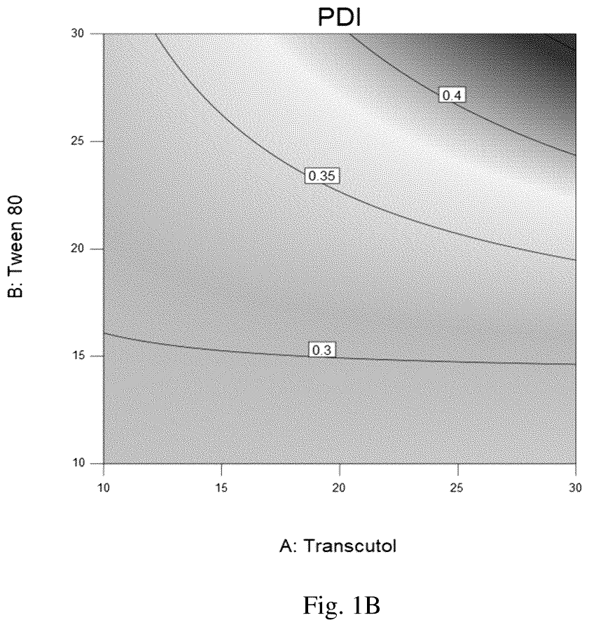

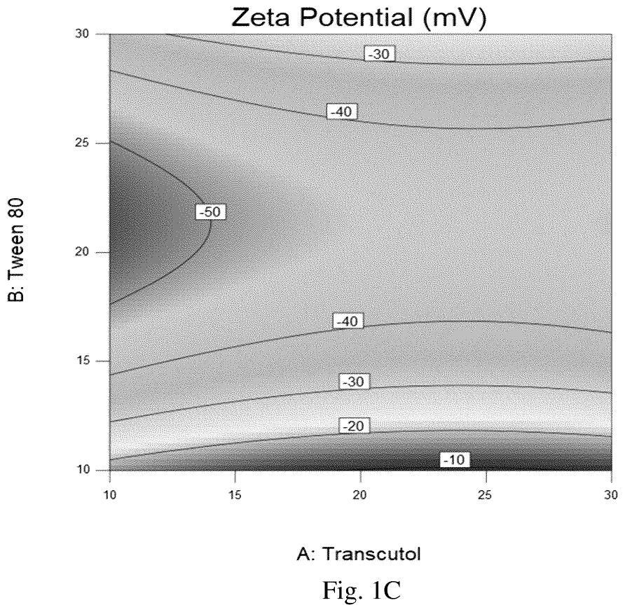

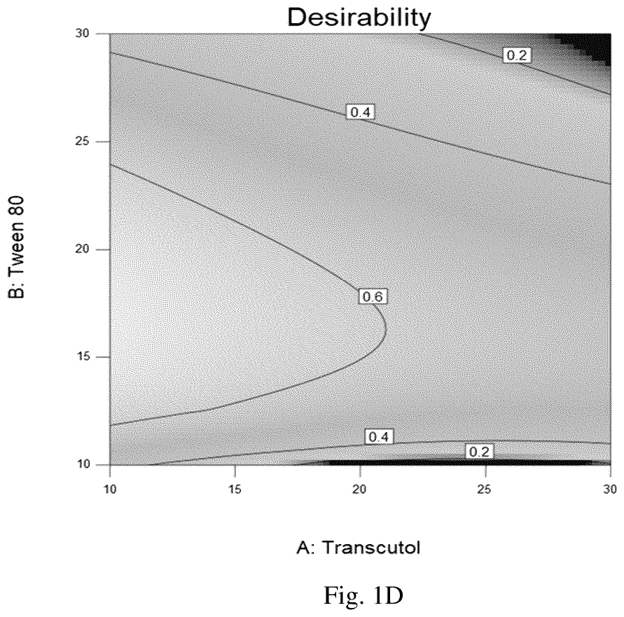

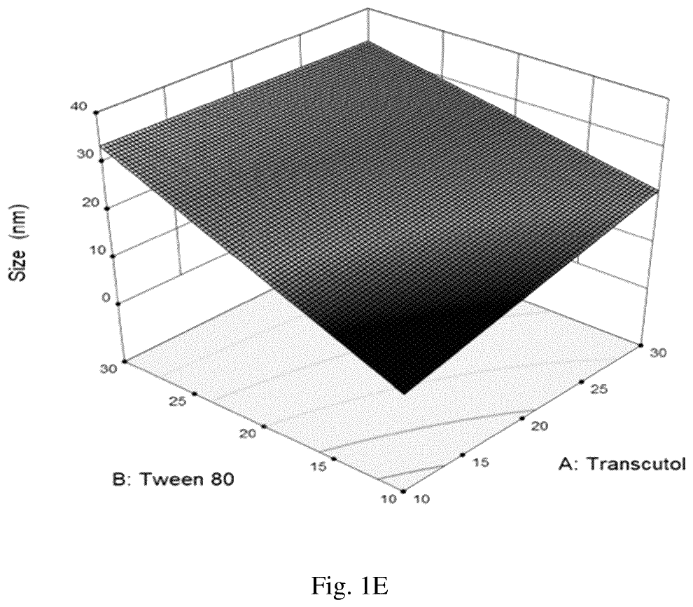

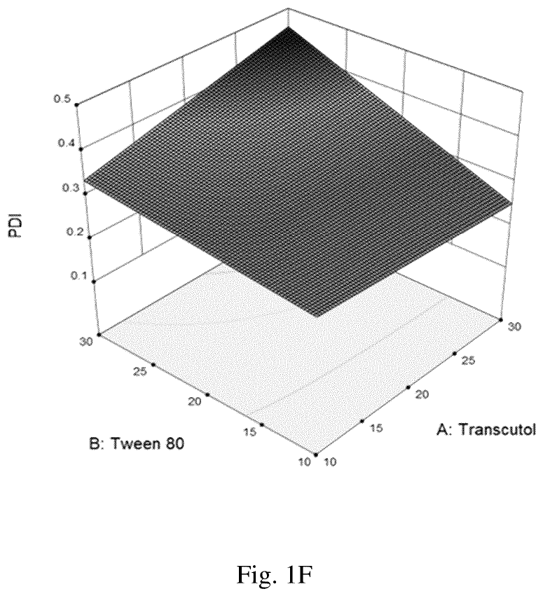

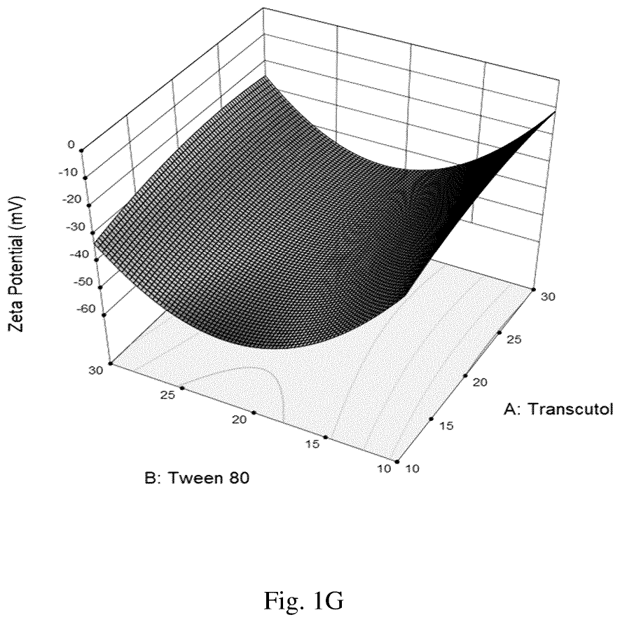

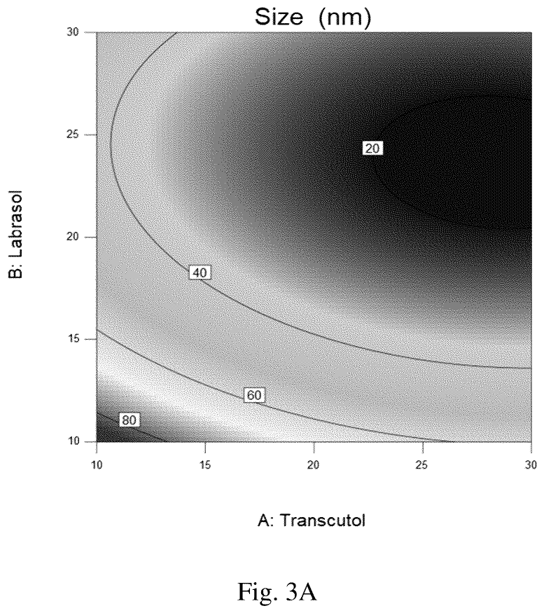

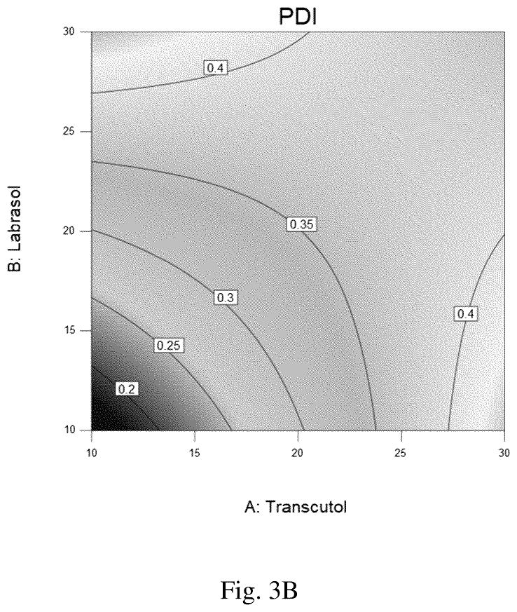

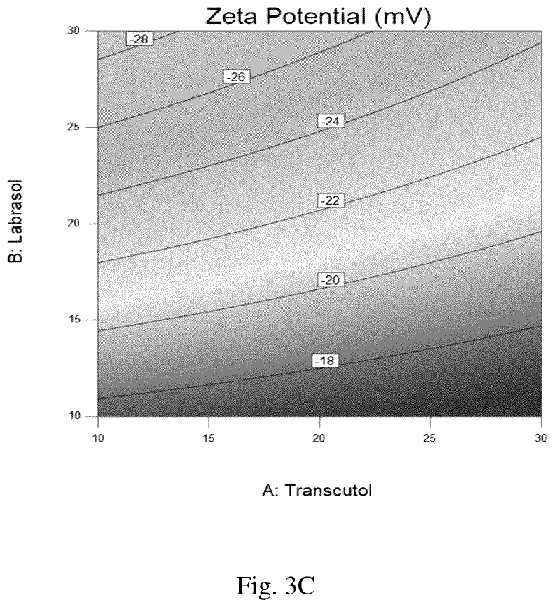

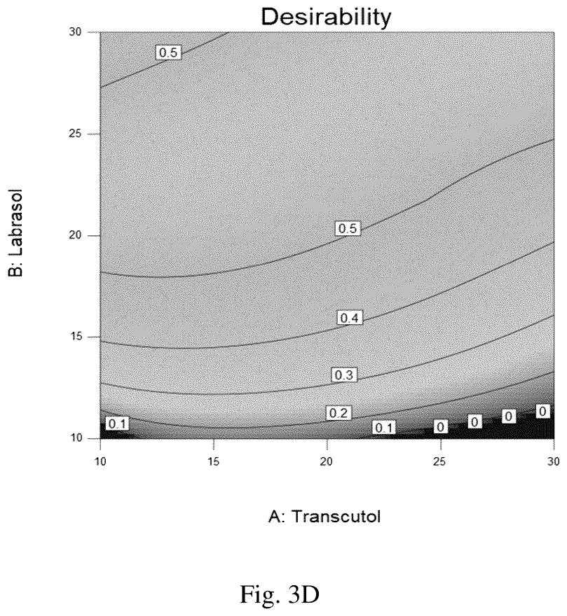

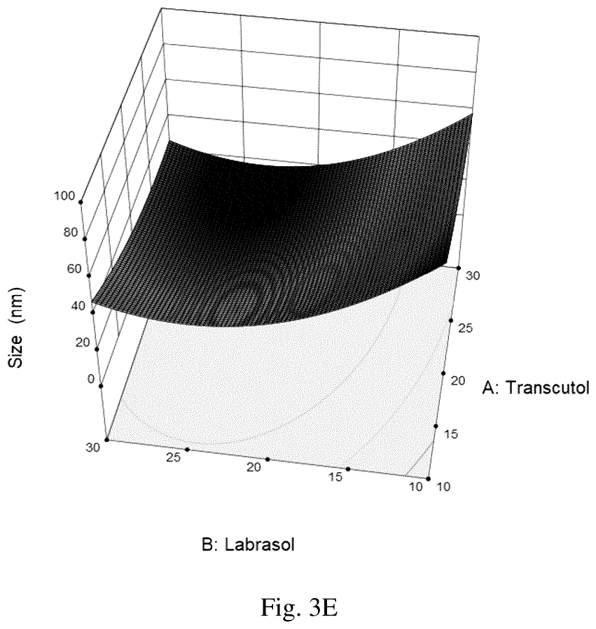

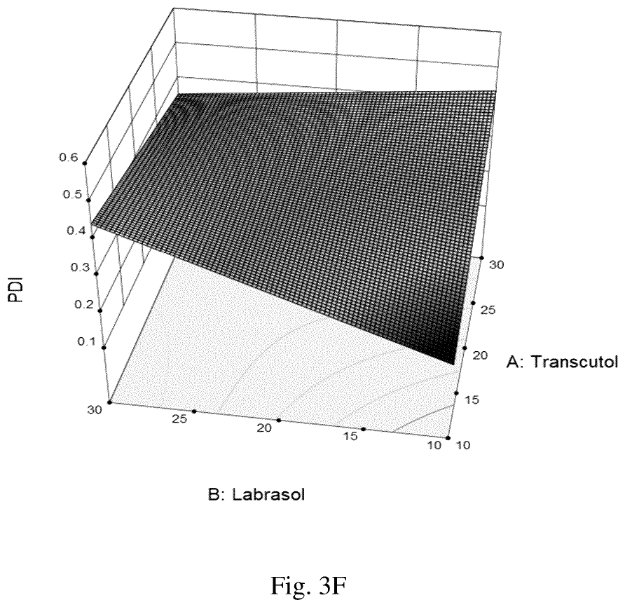

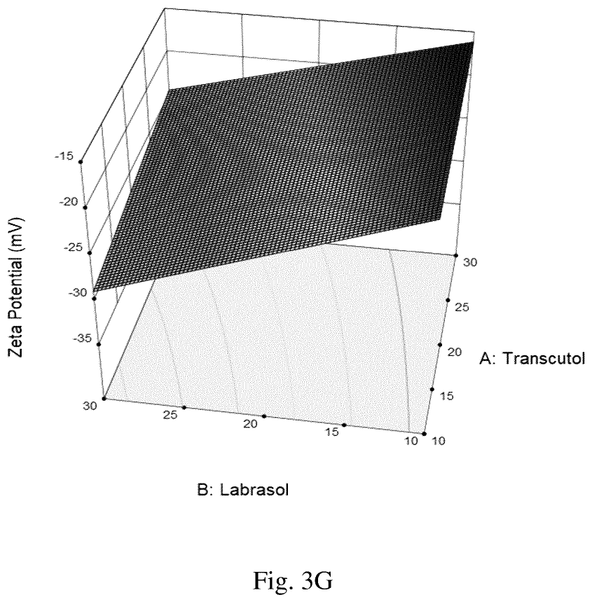

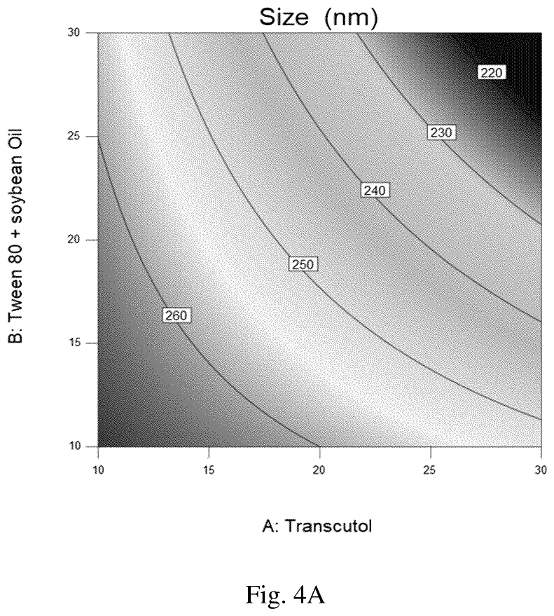

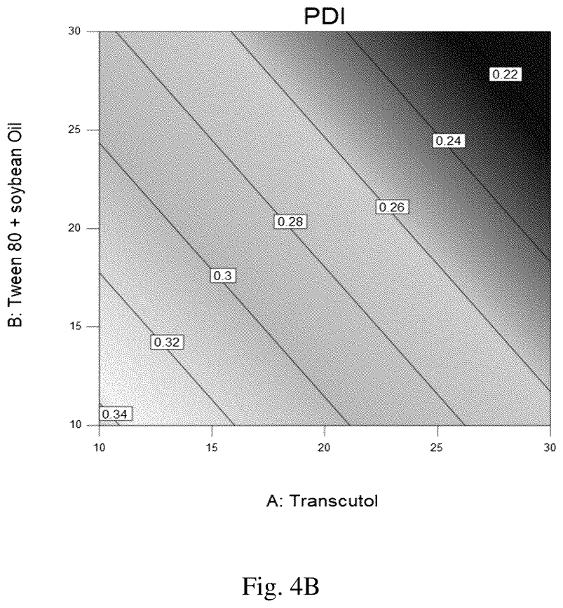

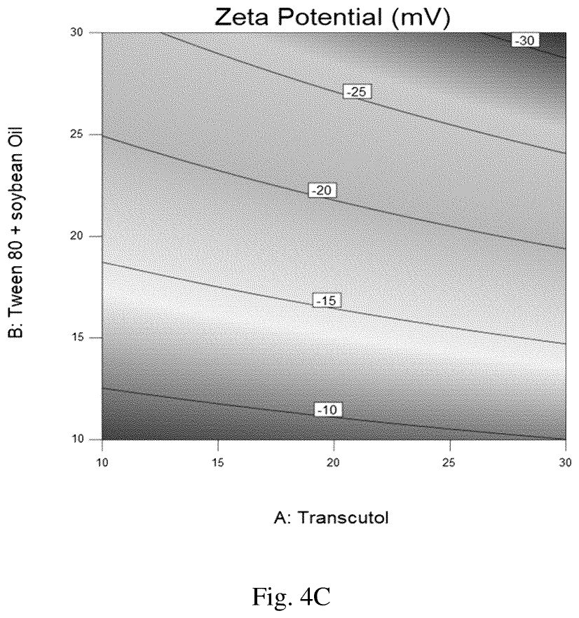

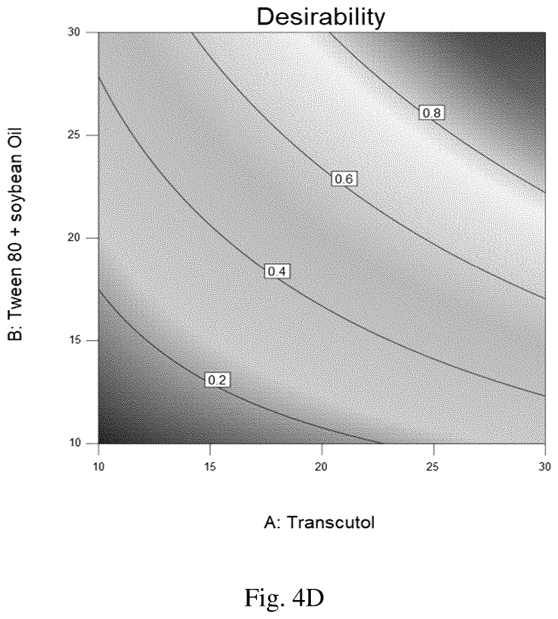

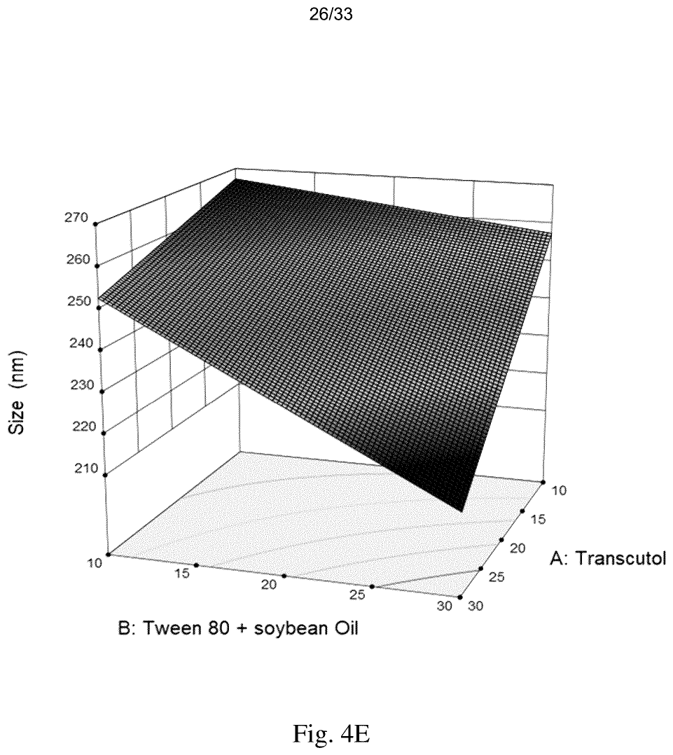

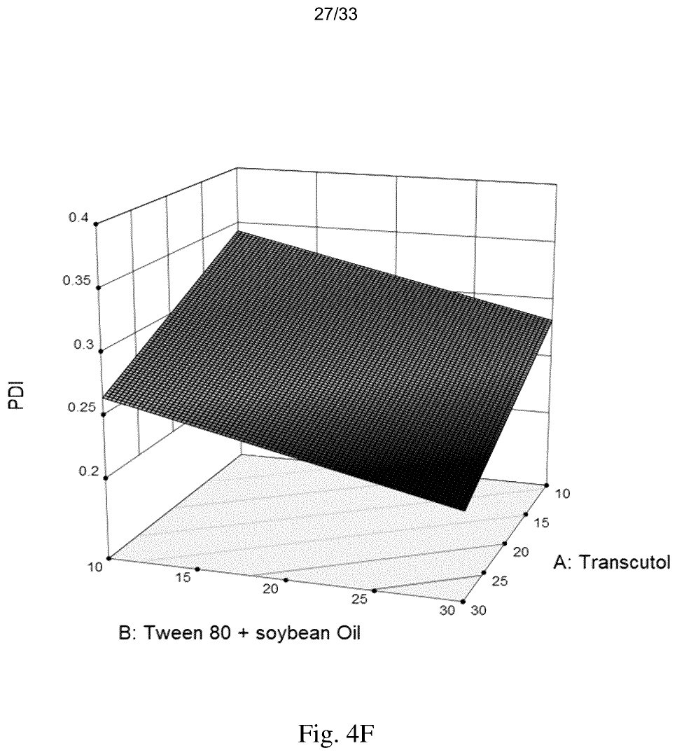

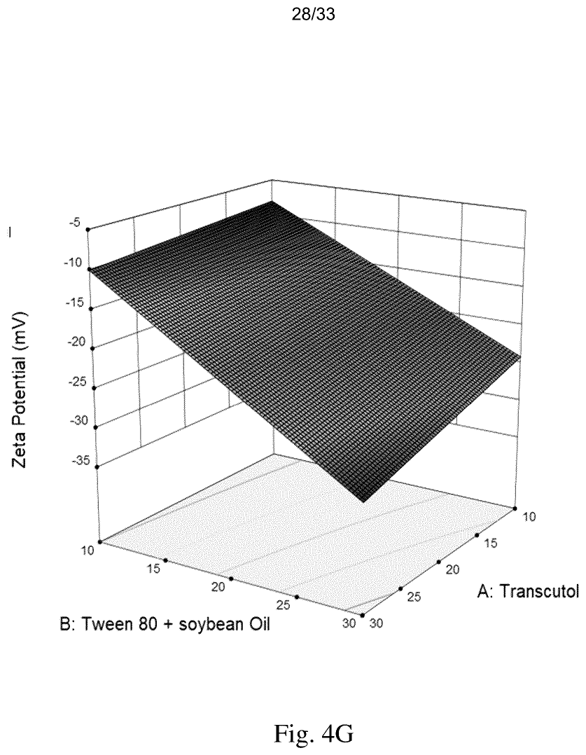

The independent factors effects was checked for the dependent variables (3 responses) using 3D response surface and contour plot (FIGS. 1A to 4G) by Design Expert Software®. The quadratic effect was to utilize for all parameters because have a good impact or effect both separately and in combination. Individual responses of analysis of variance (ANOVA) was managed by Design-Expert software, then the outcome indicated model fitting for all dataset. The independent factors: surfactant concentration (X1) and cosurfactant concentration (X2) were evaluated at three levels (low −1, medium 0, and high +1) to formulate RR nanoemulsion. Surfactant concentration (X1) was use in different concentrations such as 10% w/w, 20% w/w and 30% w/w. Cosurfactant concentration has three different concentrations such as 10% w/w, 20% w/w and 30% w/w. A total of 13 formulations each group were prepared, have 4 different groups.

TABLE 3
Independent factors variables

| Independent | | Levels* | | |
|---|---|---|---|---|
| variables | Symbols | −1 | 0 | 1 |
| Surfactant | X1 | 10 | 20 | 30 |
| Cosurfactant | X2 | 10 | 20 | 30 |

*−1 = Minimum level,
0 = medium level,
1 = Maximum level

TABLE 4
Different set of experiment in 13 trial runs in 4 groups

| | Formula | Surfactant % | Cosurfactant % | |
|---|---|---|---|---|
| Runs | (Groups) | (% w/w) | (% w/w) | Oil |
| 1 | F and H | 10 | 10 | Labrafac ™ |
| 2 | F and H | 20 | 10 | Labrafac ™ |
| 3 | F and H | 30 | 10 | Labrafac ™ |
| 4 | F and H | 10 | 20 | Labrafac ™ |
| 5 | F and H | 20 | 20 | Labrafac ™ |
| 6 | F and H | 30 | 20 | Labrafac ™ |
| 7 | F and H | 10 | 30 | Labrafac ™ |
| 8 | F and H | 20 | 30 | Labrafac ™ |
| 9 | F and H | 30 | 30 | Labrafac ™ |
| 10 | F and H | 20 | 20 | Labrafac ™ |
| 11 | F and H | 20 | 20 | Labrafac ™ |
| 12 | F and H | 20 | 20 | Labrafac ™ |
| 13 | F and H | 20 | 20 | Labrafac ™ |
| 1 | G and J | 10 | 10 | Soybean |
| 2 | G and J | 20 | 10 | Soybean |
| 3 | G and J | 30 | 10 | Soybean |
| 4 | G and J | 10 | 20 | Soybean |
| 5 | G and J | 20 | 20 | Soybean |
| 6 | G and J | 30 | 20 | Soybean |
| 7 | G and J | 10 | 30 | Soybean |
| 8 | G and J | 20 | 30 | Soybean |
| 9 | G and J | 30 | 30 | Soybean |
| 10 | G and J | 20 | 20 | Soybean |
| 11 | G and J | 20 | 20 | Soybean |
| 12 | G and J | 20 | 20 | Soybean |
| 13 | G and J | 20 | 20 | Soybean |

TABLE 5
Design of experiment for Response surface method
with 13 trial runs with results of responses

| | Mix | Responses | | | | Mix | Responses | | |
|---|---|---|---|---|---|---|---|---|---|
| Runs | Ratio (%) | Y1 | Y2 | Y3 | Runs | Ratio (%) | Y1 | Y2 | Y3 |
| F1 | 10:10 | 13.71 | 0.282 | −14.9 | G1 | 10:10 | 200.4 | 0.053 | −89.2 |
| F2 | 20:10 | 13.41 | 0.183 | −10.2 | G2 | 20:10 | 96.66 | 0.667 | −52.6 |
| F3 | 30:10 | 19.13 | 0.272 | −12.1 | G3 | 30:10 | 123.4 | 0.265 | −42.3 |
| F4 | 10:20 | 13.97 | 0.439 | −58.6 | G4 | 10:20 | 195.3 | 0.252 | −27.3 |
| F5 | 20:20 | 22.50 | 0.303 | −45.2 | G5 | 20:20 | 223.8 | 0.414 | −54.5 |
| F6 | 30:20 | 36.94 | 0.441 | −44.8 | G6 | 30:20 | 183.2 | 0.216 | −75.7 |
| F7 | 10:30 | 35.50 | 0.282 | −29.4 | G7 | 10:30 | 175.2 | 0.211 | −69.5 |
| F8 | 20:30 | 39.88 | 0.418 | −27.3 | G8 | 20:30 | 182.8 | 0.179 | −96.7 |
| F9 | 30:30 | 26.92 | 0.425 | −24.9 | G9 | 30:30 | 233.7 | 0.430 | −95.3 |
| F10 | 20:20 | 22.13 | 0.356 | −45.5 | G10 | 20:20 | 116.4 | 0.271 | −45.1 |
| F11 | 20:20 | 23.86 | 0.313 | −45.2 | G11 | 20:20 | 115.1 | 0.261 | −43.1 |
| F12 | 20:20 | 21.15 | 0.311 | −45.3 | G12 | 20:20 | 118.0 | 0.274 | −41.0 |
| F13 | 20:20 | 22.11 | 0.302 | −45.1 | G13 | 20:20 | 116.0 | 0.270 | −40.7 |
| H1 | 10:10 | 85.29 | 0.173 | −22.7 | J1 | 10:10 | 267.63 | 0.376 | −7.39 |
| H2 | 20:10 | 74.93 | 0.23 | −16.7 | J2 | 20:10 | 259.70 | 0.327 | −10.23 |
| H3 | 30:10 | 54.18 | 0.433 | −16.6 | J3 | 30:10 | 255.93 | 0.262 | −10.87 |
| H4 | 10:20 | 55.83 | 0.35 | −17.3 | J4 | 10:20 | 257.97 | 0.304 | −11.47 |
| H5 | 20:20 | 22 | 0.335 | −19.3 | J5 | 20:20 | 248.60 | 0.274 | −19.77 |
| H6 | 30:20 | 32.65 | 0.504 | −23.6 | J6 | 30:20 | 221.63 | 0.241 | −13.21 |
| H7 | 10:30 | 42.17 | 0.399 | −33 | J7 | 10:30 | 263.53 | 0.291 | −23.63 |
| H8 | 20:30 | 43.8 | 0.469 | −28.6 | J8 | 20:30 | 222.40 | 0.257 | −28.77 |
| H9 | 30:30 | 19.8 | 0.287 | −23.7 | J9 | 30:30 | 219.13 | 0.234 | −32.33 |
| H10 | 20:20 | 22 | 0.317 | −19.7 | J10 | 20:20 | 248.00 | 0.224 | −20.03 |
| H11 | 20:20 | 22 | 0.321 | −19.9 | J11 | 20:20 | 248.53 | 0.276 | −19.57 |
| H12 | 20:20 | 22.1 | 0.341 | −19.9 | J12 | 20:20 | 248.50 | 0.225 | −20.57 |
| H13 | 20:20 | 22.34 | 0.38 | −19.7 | J13 | 20:20 | 248.97 | 0.274 | −20.67 |

Y1 = Size (Nm),
Y2 = PDI,
Y3 = Zeta Potential (mV)

The particle size distribution of the RR nanoemulsion prepared is shown in table 5 and includes sizes ranging for Group F for size (13.41 to 39.88 nm), PDI (0.183 to 0.441), zeta potential (−10.2 to −58.6 mV), group G for size (96.66 to 233.70 nm), PDI (0.053 to 0.667), zeta potential (−27.3 to −96.7 mV), group H for size (19.80 to 85.29 nm), PDI (0.173 to 0.504), zeta potential (−16.6 to −33.0mV) and group I for size (219.13 to 267.63 nm), PDI (0.224 to 0.376), zeta potential (−7.39 to −32.33 mV).

The particle size of nanoemulsion is directly proportional kind of oil phase (dispersed phase in o/w emulsion). The particle size was increase attributed due to the competition of the oil particles for the emulsifying agent that remains in chamber for a limited amount. The PDI value varies from 0.0 to 1.0 and the closer to the value 0 the more homogeneous the particle distribution. The obtained PDI value of RR nanoemulsion is <1 which indicates a homogeneous droplet size distribution for all of the group formula. For zeta potential result, the higher value of the zeta potential is the more stable of the nanoemulsion system formed will prevent flocculation in the RR nanoemulsion made. These results indicate that the emulsion system formed has good stability so that no flocculation is formed. A zeta potential value of more than ±30 mV indicates moderate stability of the colloid system, where the emulsion system formed does not occur flocculation or forms aggregates and shows high stability (complete result can see in table 5).

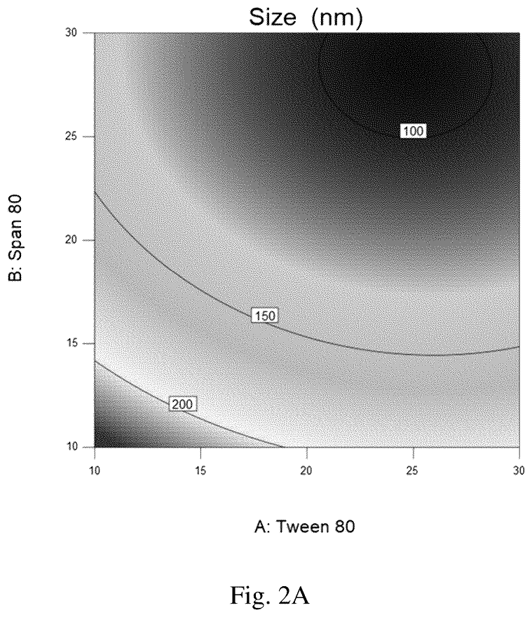

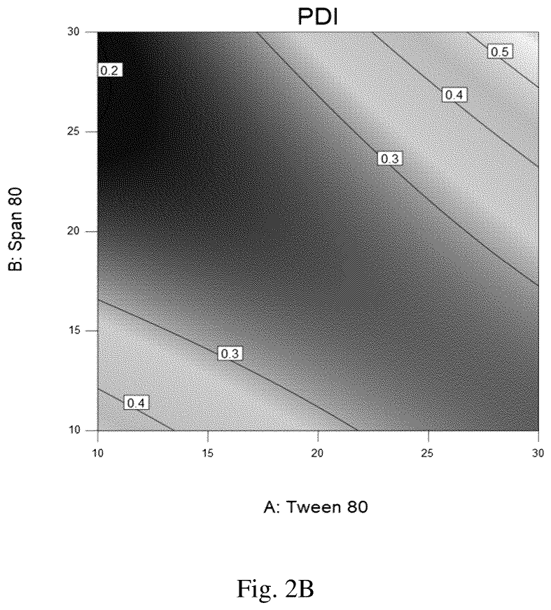

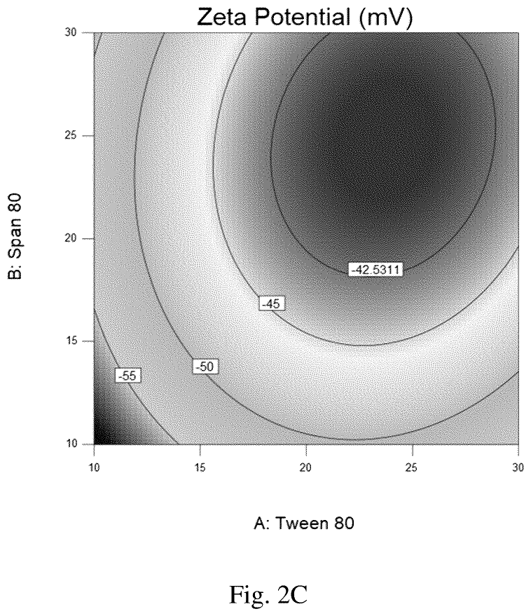

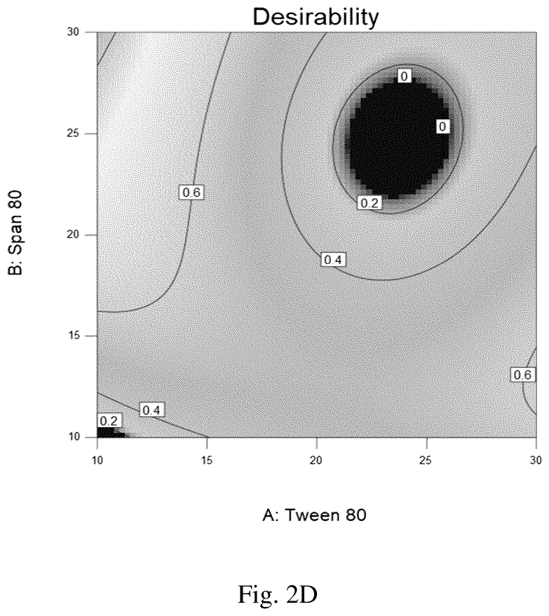

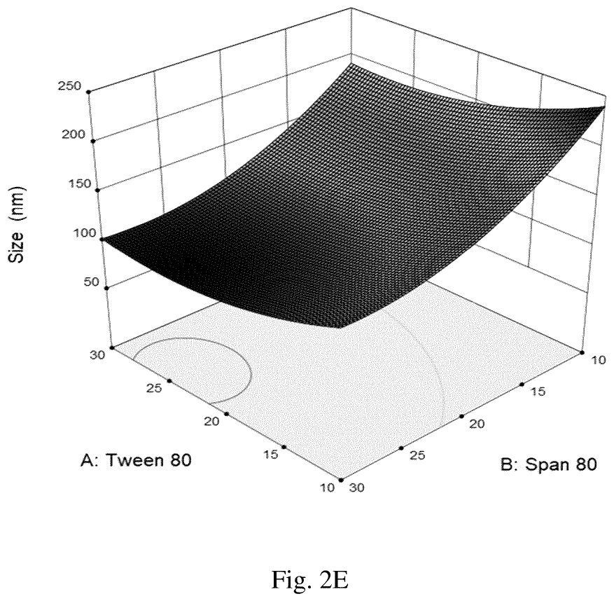

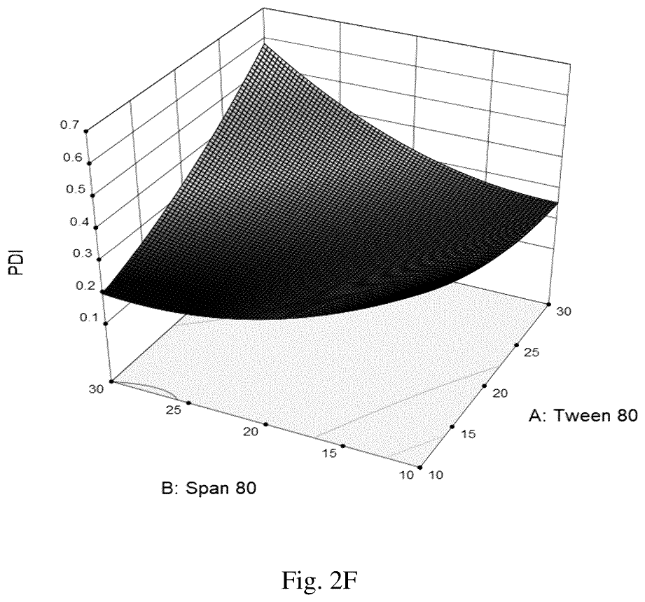

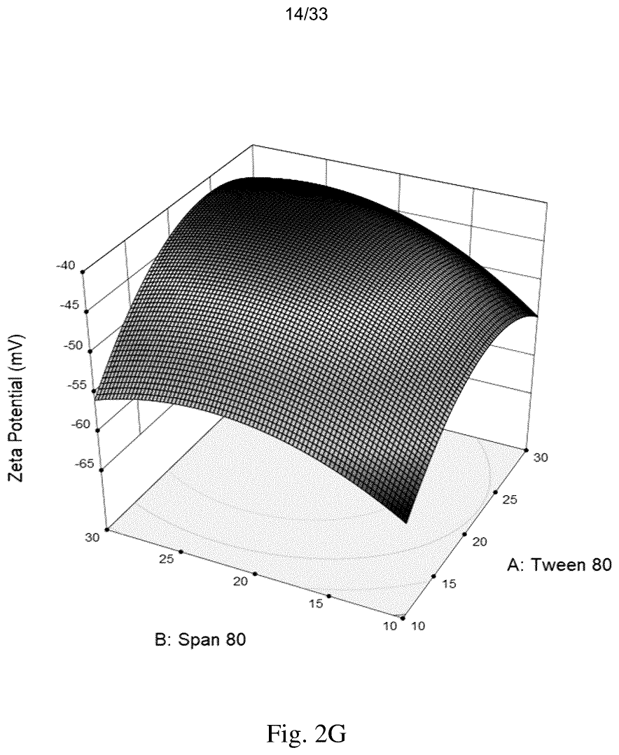

Contour plot and three-dimensional graph for effect of percentage combination surfactant and cosurfactant in three responses, such as globule size, PDI and zeta potential and also for desirability (FIGS. 1A to 4G). The area with red color indicates the highest value for each response (globule size, PDI and zeta potential), followed by yellow, green and blue color which indicates the lowest value for each response. The selection of the optimum formula can be seen in FIGS. 2D, 3D, 4D and 5D and based on the desirability value of each group, the desirability result of group F is 0.722, group G is 0.817, group H is 0.766 and group J is 0.987. The closer the desirability value is to 1, the suggested solution formula can achieve the optimal formula as desired. The closer the value is to 1, the more predicted it will be closer to the result between the predicted value and the experimental result value for the optimum formula.

### Example 3 pH Test

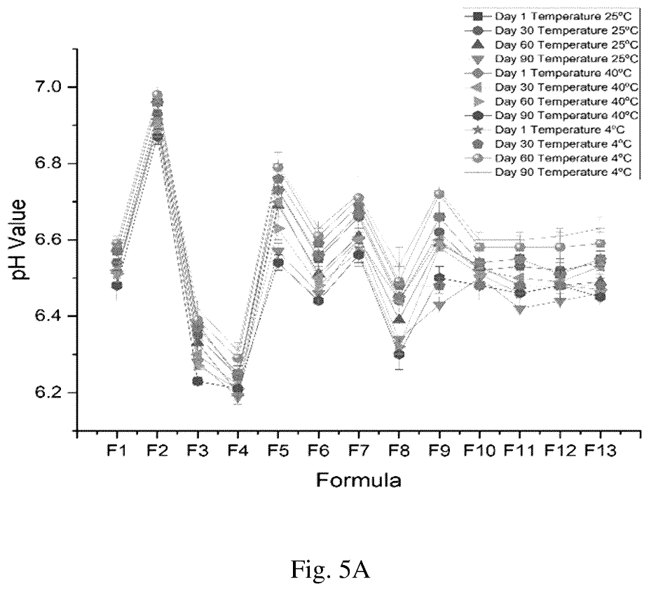

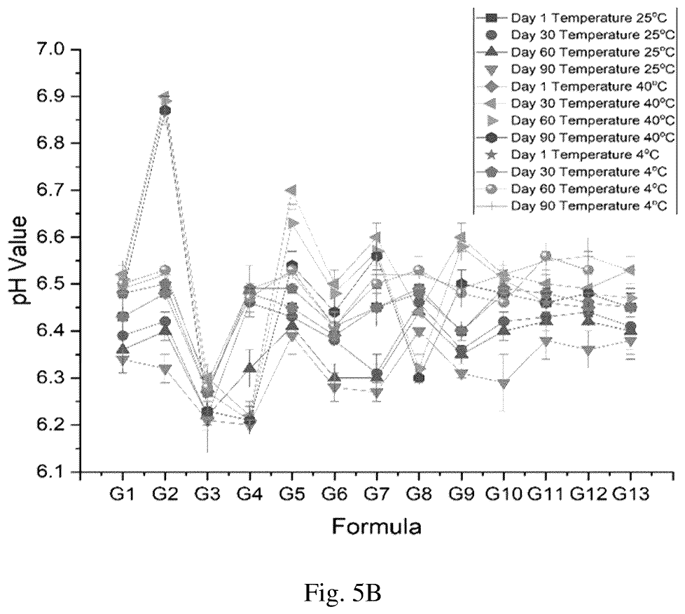

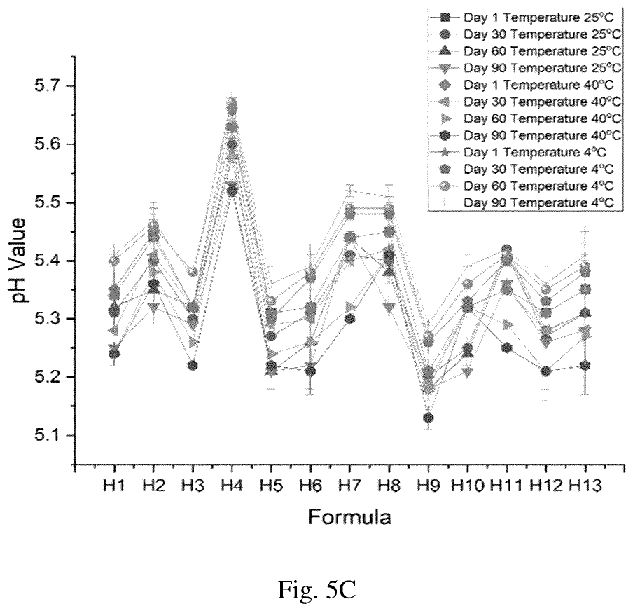

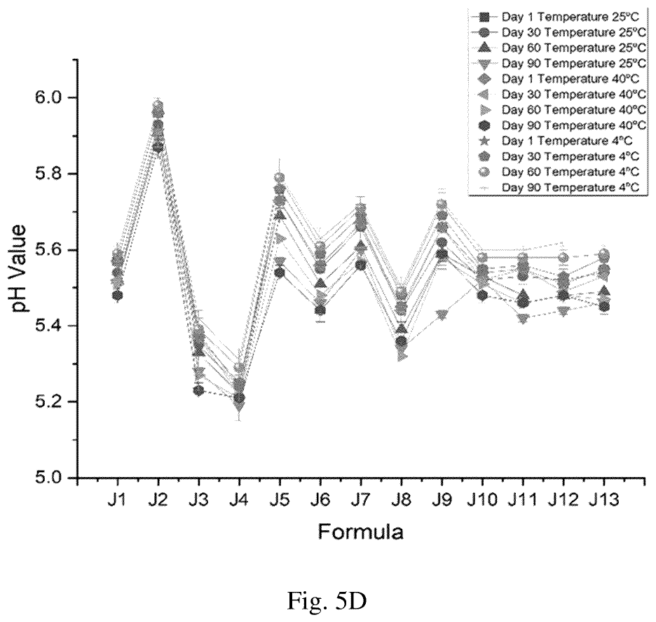

The groups are the same as example 2, the pH of both blank and drug-loaded nanoemulsions were measured with a pH meter at 25° C. Measurements were carried out in 3 different temperatures: 4° C., 25° C. and 40° C. for 90 days triplicate and data are expressed as mean±SD. The measurement of pH value was checked in day 1, day 30, day 60, day 90. As shown in FIGS. 5A to 5D, range of pH value for group F is 6.19-6.96, G is 6.21-6.56, H is 5.21-5.98 and Group J is 5.19-5.98. Requirement for topical or skin administration in pH range 4-8, if the pH value of the preparation was lower it can irritate the skin and if the pH value was high it will make the skin become dry.

### Example 4 Centrifugation Test

The groups are the same as example 2, the formulated nanoemulsions were centrifuged for 30 min at 5000 rpm and observed for phase separation, creaming and cracking. The nanoemulsions should have maximum stability, which is not a phase separation (creaming and cracking). Successful formulations were exposed to other thermodynamic stability tests. The measurements were performed in triplicate.

TABLE 6
Kinetic stability RR Nanoemulsion

| | Stirring | Phase | | |
|---|---|---|---|---|
| Runs/Group | Speed*/Time | Separation | Flocculation | Creaming |
| F1-F13 | | ND | ND | ND |
| G1-G13 | 5000 rpm/ | ND | ND | ND |
| H1-H13 | 30 minutes | ND | ND | ND |
| J1-J13 | | ND | ND | ND |

ND: Not Detected,
*revolutions per minutes

As shown in table 6, the RR nanoemulsions were checked and there were no phase separation for groups F, G, H and J, and all of the RR nanoemulsion formulations were found stable. Thus, the physical properties of nanoemulsions did not change after the centrifugation test and showed the nanoemulsion have long-term stability.

### Example 5 Freeze and Thaw Cycle Test

The groups are the same as example 2, the RR nanoemulsions were check to a freeze-thaw cycle use incubator in 40° C. and 25° C. in 6 cycles (12 days), and it was observed that nanoemulsions for groups G, H and J showed no phase separation after passing the freeze and thaw test, the prepared nanoemulsion was subjected to a Freeze and thaw cycle to determine the thermodynamic stability.

TABLE 7
Freeze and thaw test result

| Runs/ | Cycle 1 | | Cycle 2 | | Cycle 3 | | Cycle 4 | | Cycle 5 | | Cycle 6 | | |
|---|---|---|---|---|---|---|---|---|---|---|---|---|---|
| Group | 25° C. | 40° C. | 25° C. | 40° C. | 25° C. | 40° C. | 25° C. | 40° C. | 25° C. | 40° C. | 25° C. | 40° C. | Notes |
| F1-F13 | ND | ND | ND | ND | ND | ND | ND | ND | ND | ND | ND | ND | S |
| G1-G13 | ND | ND | ND | ND | ND | ND | ND | ND | ND/D* | ND/D* | ND/D* | ND/D* | G1* |
| H1-H13 | ND | ND | ND | ND | ND | ND | ND | ND | ND | ND | ND | ND | S |
| J1-J13 | ND | ND | ND | ND | ND | ND | ND | ND | ND | ND | ND | ND | S |

ND: not detected,
D: detected,
S: Stable,
*Formulation not stable

As shown in table 7 above, it was observed that after freeze and thaw cycles no creaming and no phase separation of nanoemulsions (F1-F13, G2-G13, H1-H13, J1-J13) occur, which confirmed that nanoemulsion is thermodynamically stable. For Group G1, it is stable in cycle 1 to cycle 4 and has separation in cycle 5 to cycle 6, and it was unstable.

### Example 6 Validation of the Model for Optimized RR Nanoemulsions

After inputting the independent variables and dependent variables of the four groups in Example 2 into the Design Expert® software, the four groups of optimized formulas were output. Multiple response optimization conditions would be considered if the optimization criteria produced the smallest droplet globule size, the narrowest PDI, and the zeta potential value as dependent variable responses. Based on the statistical analysis of the different experimental combinations ratio of surfactant and cosurfactant with the higher desirability point as a predicted solutions from Design Expert® software, for group F of optimization formula (Opt F) the percentage of surfactant and cosurfactant (10% of Transcutol®, 16.63% of tween 80), this solution resulted in optimal response values of 16.08 nm, 0.301 and −47.69 mV for size, PDI and zeta potential. For group G of optimized formula (Opt G) the percentage of surfactant and cosurfactant (10% of tween 80, 29.87% of span 80), this solution resulted in optimal response values of 136.97, 0.200 and −42.629 mV for size, PDI and zeta potential. For group H of optimized formula (Opt H) the percentage of surfactant and cosurfactant (28.41% of Transcutol®, 16.63% of Labrasol®), this solution resulted in optimal response values of 26.64 nm, 0.366 and −24.608 mV for size, PDI and zeta potential. Last group is group J for optimization formula (Opt J) the percentage of surfactant and cosurfactant (29.608% of Transcutol®, 30% of tween 80), this solution resulted in optimal response values of 210.34 nm, 0.366 and −31.341 mV for size, PDI and zeta potential. Next, the four groups of optimized formulations suggested by the software were tested in detail, and the experimental results are shown in Table 8.

TABLE 8
Result of validation optimization formula

| | | Responsible | Predicted | Experimental | Error |
|---|---|---|---|---|---|
| Group | composition | Variable | value | value (±SD) | (%) |
| Opt F | Transcutol ®, | Size (nm) | 16.08 | 14.88 ± 0.34 | 7.40 |
| | Tween 80, | PDI | 0.301 | 0.306 ± 0.005 | 1.66 |
| | Labrafac ™ | Zeta Potential | −47.63 | −45.70 ± 0.46 | 4.05 |
| | | (mV) | | | |
| Opt G | Tween 80, | Size (nm) | 136.97 | 129.37 ± 0.76 | 5.50 |
| | Span 80, | PDI | 0.200 | 0.203 ± 0.002 | 1.00 |
| | Soybean | Zeta Potential | −55.62 | −53.07 ± 0.90 | 4.58 |
| | | (mV) | | | |
| Opt H | Transcutol ®, | Size (nm) | 26.64 | 29.18 ± 1.74 | 9.52 |
| | Labrasol ® | PDI | 0.366 | 0.369 ± 0.008 | 0.80 |
| | Labrafac ™ | Zeta Potential | −24.608 | −26.63 ± 1.51 | 8.22 |
| | | (mV) | | | |
| Opt J | Transcutol ®, | Size (nm) | 210.34 | 220.30 ± 1.73 | 4.70 |
| | Tween 80, | PDI | 0.219 | 0.221 ± 0.004 | 1.00 |
| | Soybean | Zeta Potential | −31.341 | −32.38 ± 0.83 | 3.31 |
| | | (mV) | | | |

Result is as shown in table 8, % error=(Experimental value−Predicted value)/Experimental value×100%, difference within 10% are acceptable. Therefore, follow-up experiments were carried out for the optimized four groups' formulations.

### Example 7 Stability Study for Optimized RR Nanoemulsion

According to optimized four groups of formulas in example 6, stability test or stability studies are conducted to determine the drug substances' stability under various environmental conditions such as temperature, light, and humidity. Check the effect of external factors on the quality of a drug substance, physical formulation or formulated product is evaluated in stability test. For the stability test of optimized RR nanoemulsion can be carried out different time and temperature.

TABLE 9
Stability study of optimized RR nanoemulsion at different temperatures
for day 1, day 30, day 60, and day 90

| | | pH value | | | |
|---|---|---|---|---|---|
| Temperature | Formula | Day 1 | Day 30 | Day 60 | Day 90 |
| 25° C. | Opt F | 6.25 ± 0.02 | 6.24 ± 0.01 | 6.20 ± 0.03 | 6.19 ± 0.01 |
| 4° C. | | 6.25 ± 0.03 | 6.30 ± 0.01 | 6.27 ± 0.01 | 6.23 ± 0.01 |
| 40° C. | | 6.25 ± 0.02 | 6.38 ± 0.01 | 6.39 ± 0.01 | 6.42 ± 0.02 |
| 25° C. | Opt G | 6.27 ± 0.02 | 6.22 ± 0.06 | 6.22 ± 0.02 | 6.21 ± 0.02 |
| 4° C. | | 6.27 ± 0.02 | 6.30 ± 0.01 | 6.27 ± 0.03 | 6.21 ± 0.03 |
| 40° C. | | 6.27 ± 0.02 | 6.27 ± 0.06 | 6.28 ± 0.01 | 6.29 ± 0.04 |
| 25° C. | Opt H | 5.21 ± 0.02 | 5.20 ± 0.02 | 5.18 ± 0.02 | 5.18 ± 0.02 |
| 4° C. | | 5.21 ± 0.02 | 5.19 ± 0.02 | 5.18 ± 0.02 | 5.13 ± 0.02 |
| 40° C. | | 5.21 ± 0.02 | 5.26 ± 0.02 | 5.27 ± 0.02 | 5.29 ± 0.01 |
| 25° C. | Opt J | 5.51 ± 0.01 | 5.54 ± 0.02 | 5.49 ± 0.01 | 5.46 ± 0.03 |
| 4° C. | | 5.51 ± 0.01 | 5.49 ± 0.01 | 5.48 ± 0.01 | 5.48 ± 0.02 |
| 40° C. | | 5.51 ± 0.01 | 5.53 ± 0.02 | 5.58 ± 0.02 | 5.62 ± 0.01 |

In this study was checked for visible observation and physical test such as are the pH value, centrifugation test, and viscosity test. The pH change were carried out for day 1, day 30, day 60 and day 90 in different temperature (25° C., 40° C. and 4° C.) and all of formula for each group showed pH value in range 5.13-6.39, meet the requirements for topical drug delivery system (table 9).

In the same time, after centrifugation test was checked for organoleptic condition, such as odor, color and texture, and no phase separation was observed after 3000 rpm (30 minutes). And then, viscosity stability test was checked for the day 90 and at the end of day 90 (table 20).

TABLE 20
Stability study of optimized RR nanoemulsion at 25° C. for viscosity test
using rheometer

| | Viscosity (mPa · s) | | Stability | |
|---|---|---|---|---|
| Formula | Before | After | index (%) | Temperature |
| Opt F | 21.27 ± 0.02 | 28.25 ± 0.02 | 67.18 | 25° C. |
| Opt G | 53.20 ± 1.88 | 59.25 ± 0.87 | 88.62 | |
| Opt H | 31.53 ± 0.19 | 35.57 ± 0.03 | 87.20 | |
| Opt J | 132.89 ± 0.02 | 147.15 ± 0.06 | 89.27 | |

The result shows that not detected for change of odor, color, texture and no phase separation and no sedimentation after the centrifugation test (figure not shown) showed for long-term stability. Then the viscosity test of each sample before and after centrifugation was checked to compare those results (table 20). For stability index respectively for Opt F 67.18%, Opt G 88.62%, Opt H 87.20% and Opt J 89.27%.

### Example 8 Morphology Study

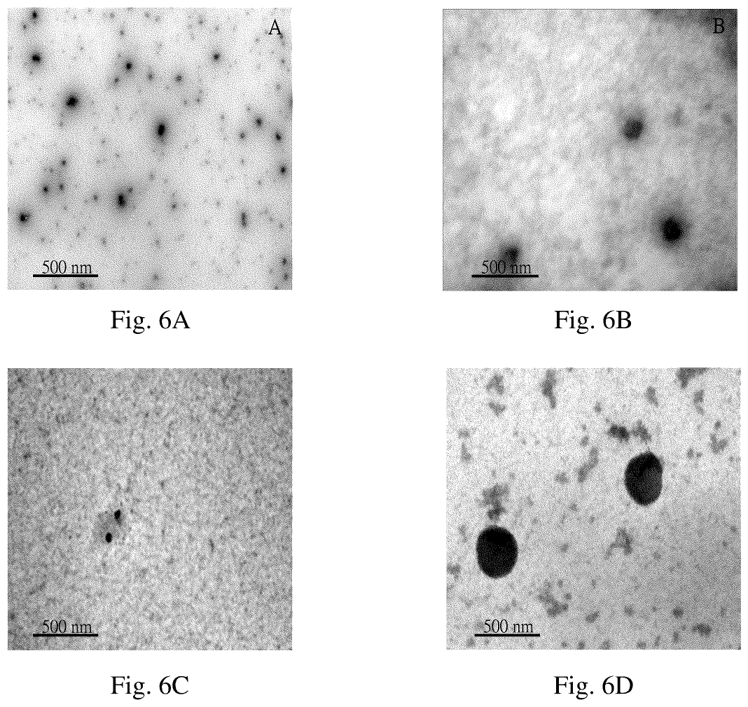

TEM images of optimized RR nanoemulsion were taken to interpret the droplet diameter and surface morphology in scale of 500 nm. The result of photomicrographs revealed spherical oil globules, with a compact nucleus surrounded by like a corona with a less intense dark color. As shown in FIGS. 6A to 6D, the morphology of the droplets of RR nanoemulsion showed clearly evident the photomicrographs that the globules were spherical, uniformly distributed, and also their size lied in close proximity to the result obtained from zetasizer. Further no have sign of coalescence of the globules or droplets was observe thus demonstrating the physical stability of RR nanoemulsion.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A Rhodiola rosea nanoemulsion, comprising:
Rhodiola rosea extract;
a surfactant comprising diethylene glycol monoethyl ester or polysorbate 80;
a cosurfactant comprising polysorbate 80, sorbitan oleate 80, or caprylocaproyl polyoxyl-8 glycerides; and
an oil comprising propylene glycol dicaprylocaprate or soybean oil, and the Rhodiola rosea extract coated in oil-in-water form.

2. The Rhodiola rosea nanoemulsion of claim 1, wherein the Rhodiola rosea extract comprises Rhodiola rosea root ethanol extract.

3. The Rhodiola rosea nanoemulsion of claim 1, wherein the Rhodiola rosea extract comprises 1,2,3,4,6-penta-O-galloyl-β-D-glucopyranoside and 1,2,3,6-tetra-O-galloyl-4-O-p-hydroxybenzoyl-β-D-glucopyranoside.

4. The Rhodiola rosea nanoemulsion of claim 1, wherein
the surfactant has a weight percentage from 10% to 30%; and
the cosurfactant has a weight percentage from 10% to 30%,
based on a total weight of the Rhodiola rosea nanoemulsion.

5. The Rhodiola rosea nanoemulsion of claim 1, wherein
the surfactant being diethylene glycol monoethyl ester has a weight percentage from 10% to 30%;
the cosurfactant being polysorbate 80 has a weight percentage from 10% to 30%, and
the oil being propylene glycol dicaprylocaprate has a weight percentage from 1% to 5%,
based on a total weight of the Rhodiola rosea nanoemulsion.

6. The Rhodiola rosea nanoemulsion of claim 5, wherein the weight percentage of the surfactant is 10%, and the weight percentage of the cosurfactant is 16.63%.

7. The Rhodiola rosea nanoemulsion of claim 1, wherein
the surfactant being polysorbate 80 has a weight percentage from 10% to 30%;
the cosurfactant being sorbitan oleate 80 has a weight percentage from 10% to 30%, and
the oil being soybean oil has a weight percentage from 1% to 5%,
based on a total weight of the Rhodiola rosea nanoemulsion.

8. The Rhodiola rosea nanoemulsion of claim 7, wherein the weight percentage of the surfactant is 10%, and the weight percentage of the cosurfactant is 29.87%.

9. The Rhodiola rosea nanoemulsion of claim 1, wherein
the surfactant being diethylene glycol monoethyl ester has a weight percentage from 10% to 30%;
the cosurfactant being caprylocaproyl polyoxyl-8 glycerides has a weight percentage from 10% to 30%, and
the oil being propylene glycol dicaprylocaprate has a weight percentage from 1% to 5%,
based on a total weight of the Rhodiola rosea nanoemulsion.

10. The Rhodiola rosea nanoemulsion of claim 9, wherein the weight percentage of the surfactant is 28.41%, and the weight percentage of the cosurfactant is 16.63%.

11. The Rhodiola rosea nanoemulsion of claim 1, wherein
the surfactant being diethylene glycol monoethyl ester has a weight percentage from 10% to 30%;
the cosurfactant being polysorbate 80 has a weight percentage from 10% to 30%, and
the oil being soybean oil has a weight percentage from 1% to 5%,
based on a total weight of the Rhodiola rosea nanoemulsion.

12. The Rhodiola rosea nanoemulsion of claim 11, wherein the weight percentage of the surfactant is 29.61%, and the weight percentage of the cosurfactant is 30%.

13. The Rhodiola rosea nanoemulsion of claim 1, wherein the Rhodiola rosea nanoemulsion has a size from 10 nm to 500 nm, a polydispersity index from 0.173 to 0.667, and a zeta potential from −96.7 mV to −7.39 mV.

14. A method for preparing Rhodiola rosea nanoemulsion, comprising:
providing a Rhodiola rosea extract;
mixing the Rhodiola rosea extract and an oil to obtain an oil phase component;
mixing a surfactant and a cosurfactant to obtain a water phase component;
adding the oil phase component into the water phase component to form oil-in-water until emulsified to obtain the Rhodiola rosea nanoemulsion,
wherein the surfactant comprises diethylene glycol monoethyl ester or polysorbate 80,
wherein the cosurfactant comprises polysorbate 80, sorbitan oleate 80, or caprylocaproyl polyoxyl-8 glycerides,
wherein the oil comprises propylene glycol dicaprylocaprate, soybean oil,
wherein a combination that when the surfactant is the diethylene glycol monoethyl ester, the cosurfactant is the sorbitan oleate 80, and the oil is soybean oil is excluded.

15. The method of claim 14, wherein the step of adding the oil phase component into the water phase component comprises dropping the oil phase component into the water phase component.

16. The method of claim 14, wherein in the step of adding the oil phase component into the water phase component, forming oil-in-water until emulsified is by ultrasonic vibration.

17. The method of claim 14, wherein
the surfactant has a weight percentage from 10% to 30%; and
the cosurfactant has a weight percentage from 10% to 30%,
based on a total weight of the Rhodiola rosea nanoemulsion.
